(19) United States
(12) Patent Application Publication
(10) Pub. No.: US 2025/0195740 A1
(43) Pub. Date: Jun. 19, 2025

(54) SUCTION AND ASPIRATION COLLECTION DEVICE

(71) Applicant: MicroVention, Inc., Aliso Viejo, CA (US)

(72) Inventors: David Kim, Mission Viejo, CA (US); Joseph Rye, Rancho Santa Margarita, CA (US); Hari Raman, Aliso Viejo, CA (US)

(21) Appl. No.: 18/849,398

(22) PCT Filed: Mar. 22, 2023
(86) PCT No.: PCT/US2023/064800

§ 371 Date: Sep. 20, 2024

Related U.S. Application Data
(60) Provisional application No. 63/322,487, filed on Mar. 22, 2022.

(51) Int. Cl. A61M 1/00

(52) U.S. Cl. CPC A61M 1/60; A61M 2205/583; A61M 2205/7545

(57) ABSTRACT

A suction and aspiration collection device may be provided by collection device, comprising: a fluid collector, having a first translucent wall defining an opening in a first plane; a lid, configured to selectively interface with the fluid collector to seal the opening and define a fluid containment region, the lid defining a first through-hole substantially parallel to the first plane; and a solids filter having a second translucent wall defining a plurality of fluid outlets and configured to selectively interface with the first through-hole to dispose the plurality of fluid outlets within the fluid containment region.

The present disclosure claims priority from U.S. Provisional Patent Application 63/322,487, titled “SUCTION AND ASPIRATION COLLECTION DEVICE”, filed on Mar. 22, 2022, which is incorporated herein by reference in its entirety.

### BACKGROUND

Suction and aspiration devices are used as medical devices that remove bodily fluids, such as blood, from a patient. A vacuum pump supplies negative pressure (e.g., a relative vacuum) to draw the bodily fluids and any solids carried by or with the bodily fluid into a collection device. A filter placed in the flow path of the bodily fluid into the collection device may be used to separate solids, such as blood clots, from the collected fluid.

### SUMMARY

The present disclosure is generally related to an improved suction and aspiration collection device. The improvements described herein provide various benefits, including, but not limited to: easier use of the collection device, easier analysis of fluids and solids collected by the collection device, more robust and reliable sealing of the collection device, greater pressure differential tolerances for the collection device, and improved or simplified manufacturing techniques therefore.

One embodiment of the present disclosure is a collection device, comprising: a fluid collector, having a first translucent wall defining an opening in a first plane; a lid, configured to selectively interface with the fluid collector to seal the opening and define a fluid containment region, the lid defining a first through-hole substantially parallel to the first plane; and a solids filter having a second translucent wall defining a plurality of fluid outlets and configured to selectively interface with the first through-hole to dispose the plurality of fluid outlets within the fluid containment region.

In some embodiments of the collection device, the collection device is further comprising a cap configured to: secure the solids filter in the first through-hole; and secure intake tubing into the solids filter. In some such embodiments, the lid further defines a second through-hole substantially parallel to the first plane, further comprising: vacuum tubing secured to the second through-hole configured to impart a negative pressure in the fluid containment region to draw a fluid through the intake tubing into the fluid containment region. In some such embodiments, the collection device is further comprising: a return filter disposed in the fluid containment region and connected to the second through-hole.

In some embodiments of the collection device, the collection device is further comprising a pressure film disposed in the fluid containment region, configured to change color in response to the fluid containment region losing containment after the negative pressure is imparted. In some such embodiments, the lid extends for a first distance from the first plane, the pressure film is located a second distance from the first plane, and the plurality of fluid outlets is located at least a third distance from the first plane; wherein the first distance, the second distance, and the third distance are measured perpendicularly from the first plane; and wherein the first distance is less than the second distance and the third distance is greater than the second distance.

In some embodiments of the collection device, the negative pressure is selected from the group consisting of: between 1 and 5 pounds per square inch (PSI) less than a current atmospheric pressure; 5 PSI less than the current atmospheric pressure; between 5-10 PSI less than the current atmospheric pressure; 10 PSI less than the current atmospheric pressure; between 10-15 PSI less than the current atmospheric pressure; and 15 PSI or less than the current atmospheric pressure.

In some embodiments of the collection device, the second translucent wall describes an open cylindrical prism including a collecting face, substantially parallel to the first plane, a directing face, substantially perpendicular to the first plane and connected to the collecting face on a first side and that defines, on a second side opposite to the first side, an intake opening, substantially parallel to the first plane. In some such embodiments, the plurality of fluid outlets is included in the collecting face with flow-paths substantially perpendicular to the first plane, or the plurality of fluid outlets is included in the directing face. In some such embodiments, the directing face comprises: a first portion; and a second portion; wherein the first portion is proximal to the collecting face and distal to the intake opening relative to the second portion and includes all of the plurality of fluid outlets that are included in the directing face; and wherein the second portion is distal to the collecting face and proximal to the intake opening relative to the first portion. In some such embodiments, the first portion comprises a first percentage of the directing face and the second portion comprises a second percentage of the directing face, greater than the first percentage. In some embodiments, the plurality of fluid outlets have flow-paths substantially parallel to the first plane and are substantially parallel to each other or the plurality of fluid outlets have flow-paths substantially parallel to the first plane and are radially oriented relative to the directing face.

In some embodiments, of the collection device, the solids filter includes a first section mated to a second section, wherein each of the first section and the second section define half of the open cylindrical prism. In some such embodiments, the lid defines the first through-hole non-concentrically relative to a central axis of the fluid collector and the lid substantially perpendicular to the first plane that positions the solids filter closer to one portion of the first translucent wall, wherein the lid further comprises a port alignment feature configured to engage a lip defined around the intake opening to orient a mating line where the first section contacts the second section relative to the one portion of the fluid collector.

In some embodiments of the collection device, the lid is secured to the fluid collector via a plurality of twist-locks, wherein each twist-lock is positioned at a given arc distance counterclockwise from a first neighboring twist-lock and the given arc distance clockwise from a second neighboring twist-lock. In some such embodiments, each twist-lock of the plurality of twist-locks comprises: a tab defined on an outer circumference of the first translucent wall; a ramp defined in the lid, configured to push the lid into closer contact with the fluid collector when the tab is captured on the ramp and the lid is rotated in a first direction relative to the fluid collector; and a catchment defined in the lid, configured to capture the tab after the tab is captured on the ramp and the lid is rotated a first amount in the first direction relative to the fluid collector and thereafter prevent further rotation of the lid relative to the fluid collector in the first direction or a second direction, opposite to the first direction. In some such embodiments, the tab includes a first magnet and the catchment includes a second magnet, wherein the first magnet and the second magnet are configured to magnetically attract one another when the tab is captured in the catchment.

In some embodiments of the collection device, the collection device is further comprising: a first compressible sealing gasket included in the lid and oriented concentrically to a central axis substantially perpendicular to the first plane to interface with a first mating surface of the fluid collector when the lid is selectively engaged with the fluid collector; and a second compressible sealing gasket included in the fluid collector and oriented concentrically to the central axis to interface with a second mating surface of the lid when the lid is selectively engaged with the fluid collector. In some such embodiments, the first compressible sealing gasket and the first mating surface are configured to engage one another when the lid is affixed to the fluid collector, wherein the second compressible sealing gasket and the second mating surface are configured to not engage one another when the lid is affixed to the fluid collector until a threshold negative pressure is supplied to the fluid containment region. In some such embodiments, an outer radius of the second compressible sealing gasket is smaller than an inner radius of the first compressible sealing gasket. In some such embodiments, the first mating surface is located proximal to the first plane relative to the second mating surface.

In some embodiments of the collection device, the lid is translucent.

In some embodiments of the collection device, the fluid collector includes graduations corresponding to known volumes of the fluid containment region at known heights relative to a base of the fluid collector opposite to the opening.

One embodiment of the present disclosure is a filter, comprising: a collecting face, having a circumference; a directing face, made of a translucent material, connected to the collecting face around the circumference in a first plane and defining an intake opening in a second plane substantially parallel to the first plane; a plurality of fluid outlets defined through at least one of: the collecting face; and a first portion of the directing face proximate to the first plane and distal to the second plane, wherein a second portion of the directing face proximate to the second plane and distal to the first plane is configured to direct fluid to the first portion and the collecting face.

In some embodiments of the filter, the filter comprises a first section mated with a second section, wherein first section comprises half of the collecting face and half of the directing face. In some such embodiments, the first section defines a different number, orientation, or pattern of fluid outlets of the plurality of fluid outlets from the second section. In some such embodiments, the filter is further comprising a lip bounding the directing face on an opposite side of the directing face relative to the collecting face, the lip having a greater circumference than the collecting face and defining a filter alignment feature to interface with an intake port of a lid to orient the filter relative to the lid.

One embodiment of the present disclosure is a collection device, comprising: a lid, including a plurality of female twist-lock engagement features; and a fluid collector, including a plurality of male twist-lock engagement features corresponding to the plurality of female twist-lock engagement features, wherein each paired male twist-lock engagement feature and female twist-lock engagement feature comprises: a tab defined on an outer circumference of the fluid collector; a ramp defined in the lid, configured to push the lid into closer contact with the fluid collector when the tab is captured on the ramp and the lid is rotated in a first direction relative to the fluid collector; and a catchment defined in the lid, configured to capture the tab after the tab is captured on the ramp and the lid is rotated a first amount in the first direction relative to the fluid collector and thereafter impede further rotation of the lid relative to the fluid collector in the first direction or a second direction, opposite to the first direction.

In some embodiments of the collection device, the tab is positioned at a given arc distance counterclockwise from a first neighboring tab and the given arc distance clockwise from a second neighboring tab. In some such embodiments, the tab includes a first magnet and the catchment includes a second magnet, wherein the first magnet and the second magnet are configured to magnetically attract one another when the tab is captured in the catchment. In some such embodiments, the lid includes an intake port that defines a first through-hole, the collection device further comprising: a solids filter having defining a plurality of fluid outlets and configured to selectively interface with the intake port to dispose the plurality of fluid outlets through the first through-hole and into a fluid containment region defined between the lid and the fluid collector; intake tubing; and a cap configured to secure the solids filter in the first through-hole and secure the intake tubing into the solids filter via a one-way valve. In some such embodiments, the fluid collector and the solids filter are translucent.

In some embodiments of the collection device, the collection device is further comprising: a first gasket included in the lid and oriented to interface with a first mating surface of the fluid collector when the lid is selectively engaged with the fluid collector; and a second gasket included in the fluid collector and oriented to interface with a second mating surface of the lid when the lid is selectively engaged with the fluid collector.

One embodiment of the present disclosure is a collection device, comprising: a lid including a first gasket and a first mating surface that are oriented concentrically to a central axis; and a fluid collector including a second gasket and a second mating surface that are oriented concentrically to the central axis, wherein the second gasket is located to engage with the first mating surface and the second mating surface is located to engage with the first gasket when the lid is selectively engaged with the fluid collector.

In some embodiments of the collection device, the first gasket and the first mating surface are configured to engage one another when the lid is affixed to the fluid collector, wherein the second gasket and the second mating surface are configured to not engage one another when the lid is affixed to the fluid collector until a threshold negative pressure is supplied to the fluid containment region.

In some embodiments of the collection device, an outer radius of the second compressible sealing gasket is smaller than an inner radius of the first compressible sealing gasket.

In some embodiments of the collection device, the lid includes an intake port that defines a first through-hole, the collection device further comprising: a solids filter defining a plurality of fluid outlets and configured to selectively interface with the intake port to dispose the plurality of fluid outlets through the first through-hole and into a fluid containment region defined between the lid and the fluid collector; intake tubing; and a cap configured to secure the solids filter in the first through-hole and secure the intake tubing into the solids filter via a one-way valve. In some such embodiments, the fluid collector and the solids filter are translucent.

One embodiment of the present disclosure is a collection device, comprising: a fluid collector, having a base, a first translucent wall attached to the base, and an opening in a first plane; a lid having a first engagement interface, where in an engaged configuration of the lid, the first engagement interface of the lid engages with the fluid collector to seal the opening to form a fluid containment region, wherein the lid has a first through-hole; and a solids filter having a collecting face, a second translucent wall attached to the collecting face, and includes a plurality of fluid outlets, wherein the solids filter further comprises a second engagement interface, where in an engaged configuration of the solids filter, the second engagement interface of the solids filter engages with the first through-hole such that the plurality of fluid outlets are within the fluid containment region.

In some embodiments of the collection device, the collection device further comprises: a cap having a third engagement interface, where in an engaged configuration of the cap, the third engagement interface of the cap engages the solids filter; and an intake tubing attached to the cap wherein the intake tubing is in fluid communication with the cap and the solids filter. In some embodiments, the lid further includes a second through-hole, further comprising: a vacuum tubing secured to the second through-hole to impart a negative pressure in the fluid containment region to draw a fluid through the intake tubing into the fluid containment region. In some embodiments, the collection device further comprises a return filter disposed in the fluid containment region and connected to the second through-hole. In some embodiments, the collection device further comprises a pressure film disposed in the fluid containment region, wherein the pressure film has a first color at first pressure within the fluid containment region, and wherein the pressure film has a second color, different from the first color, at a second pressure within the fluid containment region in response to the negative pressure imparted by a negative pressure source in an operation mode. In some embodiments, the lid extends for a first distance from the first plane, the pressure film is located a second distance from the first plane, and the plurality of fluid outlets is located at least a third distance from the first plane; wherein the first distance, the second distance, and the third distance are measured perpendicularly from the first plane; and wherein the first distance is less than the second distance, and the third distance is greater than the second distance.

In some embodiments of the collection device, the negative pressure is selected from the group consisting of: between 1 and 5 pounds per square inch (PSI) less than a current atmospheric pressure; 5 PSI less than the current atmospheric pressure; between 5-10 PSI less than the current atmospheric pressure; 10 PSI less than the current atmospheric pressure; between 10-15 PSI less than the current atmospheric pressure; and 15 PSI or less than the current atmospheric pressure.

In some embodiments of the collection device, the solids filter has an intake opening on an opposite end of second translucent wall from the collecting face, wherein the intake opening and the collecting face are substantially parallel to the first plane when the solids filter engages with the first through-hole. In some embodiments, the plurality of fluid outlets are included in the collecting face with flow-paths substantially perpendicular to the first plane or the plurality of fluid outlets are included in the second translucent wall. In some embodiments, the second translucent wall comprises: a first portion; and a second portion; wherein the first portion is closer to the collecting face and further from the intake opening relative to the second portion and includes all of the plurality of fluid outlets that are included in the directing face; and wherein the second portion is further from the collecting face and closer to the intake opening relative to the first portion. In some embodiments, the first portion comprises a first percentage of the second translucent wall and the second portion comprises a second percentage of the second translucent wall, greater than the first percentage. In some embodiments, the plurality of fluid outlets have flow-paths substantially parallel to the first plane and are substantially parallel to each other or have flow-paths substantially parallel to the first plane and are radially oriented relative to the second translucent wall. In some embodiments, the solids filter includes a first section mated to a second section, wherein each of the first section and the second section define half of the solids filter. In some embodiments, the first through-hole is located non-concentrically relative to a central axis of the fluid collector when the lid is engaged with the fluid collector to position the solids filter closer to one portion of the first translucent wall when the solids filter is also engaged with the lid, wherein the second engagement interface includes a lip defined around the intake opening to orient a mating line where the first section contacts the second section relative to the one portion of the fluid collector.

In some embodiments, the first engagement interface includes a plurality of twist-locks, wherein each twist-lock is positioned at a given arc distance counterclockwise from a first neighboring twist-lock and the given arc distance clockwise from a second neighboring twist-lock. In some embodiments, each twist-lock of the plurality of twist-locks comprises: a tab included on an outer circumference of the first translucent wall; a ramp included in the lid, that pushes the lid into closer contact with the fluid collector when the tab is captured on the ramp and the lid is rotated in a first direction relative to the fluid collector; and a catchment included in the lid, that captures the tab when the twist-lock is in a first state after the tab is captured on the ramp and the lid is rotated a first amount in the first direction relative to the fluid collector, wherein the catchment, when the twist-lock is in the first state, prevents further rotation of the lid relative to the fluid collector in the first direction or a second direction, opposite to the first direction. In some embodiments, the tab includes a first magnet and the catchment includes a second magnet, wherein the first magnet and the second magnet have polarities and strengths that magnetically attract one another when the tab is captured in the catchment.

In some embodiments, the collected device further comprises a first compressible sealing gasket included in the lid and oriented concentrically to a central axis substantially perpendicular to the first plane to interface with a first mating surface of the fluid collector when the lid is selectively engaged with the fluid collector; and a second compressible sealing gasket included in the fluid collector and oriented concentrically to the central axis to interface with a second mating surface of the lid when the lid is selectively engaged with the fluid collector. In some embodiments, the first compressible sealing gasket and the first mating surface engage one another when the lid is affixed to the fluid collector, wherein the second compressible sealing gasket and the second mating surface do not engage one another when the lid is affixed to the fluid collector until a threshold negative pressure is supplied to the fluid containment region. In some embodiments, an outer radius of the second compressible sealing gasket is equal to or smaller than an inner radius of the first compressible sealing gasket. In some embodiments, the first mating surface is located closer to the first plane relative to the second mating surface.

One embodiment of the present disclosure is a collection device, comprising: a pressure containment means, having a fluid containment region and wherein at least a portion of the pressure containment means is translucent; a solids filtration means, disposed within the fluid containment region, wherein the solids filtrations means is translucent; a first fluid communication means connected to the pressure containment means and separated from the fluid containment region via the solids filtration means; and a second fluid communication means connected to the pressure containment means and a negative pressure source to impart a negative pressure in the fluid containment region to draw a fluid through the first fluid communication means through the solids filtration means and into the fluid containment region.

In some embodiments, the collect device further comprises: an aerosol filtration means, disposed within the fluid containment region and connected to the second fluid communication means to reduce intake of the fluid from the fluid containment region into the second fluid communication means when the negative pressure is being imparted. In some embodiments, the solids filtration means is held in the fluid containment region via engagement features interfacing with a first through-hole in a lid of the pressure containment means, wherein the first through-hole is positioned non-concentrically to a central axis of the pressure containment means to place the solids filtration means off-center relative to the central axis. In some embodiments, the pressure containment means includes a lid and a fluid collector with a first sealing means and a second sealing means.

In some embodiments, the lid includes a plurality of first engagement means and the fluid collector includes a plurality of second engagement means that each engage with a corresponding first engagement means of the plurality of first engagement means via a downward force and a rotational force in a first direction applied to the lid when in contact with the fluid collector to seal the pressure containment means via the first sealing means. In some embodiments, the second sealing means is not engaged until the negative pressure imparted to the pressure containment means via the negative pressure source.

In some embodiments, each first engagement means of the plurality of first engagement means includes a magnet; and wherein a catchment included in each second engagement means that captures a corresponding first engagement means when the pressure containment means is sealed includes a ferromagnetic inclusion to magnetically interface with the magnet of the corresponding first engagement means. In some embodiments, each second engagement means of the plurality of second engagement means includes a magnet in a catchment that that captures a corresponding first engagement means when the pressure containment means is sealed; and wherein each first engagement means of the plurality of first engagement means includes a ferromagnetic inclusion to magnetically interface with the magnet of a corresponding second engagement means when the pressure containment means is sealed. In some embodiments, each wherein each first engagement means of the plurality of first engagement means includes a first magnet of a first polarity; and each second engagement means of the plurality of second engagement means includes a second magnet of a second plurality in a catchment that that captures a corresponding first engagement means when the pressure containment means is sealed to magnetically interface with the first magnet of the corresponding first engagement means.

In some embodiments, the collection device further comprises a pressure indicating means disposed in the fluid containment region and visible from outside of the fluid containment region.

One embodiment of the present disclosure is a method, comprising: attaching a lid to a fluid collector to form a fluid containment region with an airtight seal between the lid and the fluid collector; securing a solids filter into the fluid containment region through the lid; creating fluid communication between a negative pressure source and the fluid containment region and between the fluid containment region and a filtration target; applying negative pressure via the fluid communication to draw a fluid from the filtration target, through the solids filter, into the fluid containment region; and in response to a collection criteria being reached, disengaging the negative pressure from the fluid communication.

In some embodiments, the collection criteria is reached in response to detecting presence of solids in the solids filter, wherein the collection criteria is determined to have been reached via visual inspection through a translucent wall of the fluid collector and a translucent wall of the solids filter. In some embodiments, the collection criteria is reached in response to a change in color of a pressure film disposed in the fluid containment region, wherein the collection criteria is determined to have been reached via visual inspection through a translucent wall of the fluid collector.

In some embodiments, attaching the lid comprises: rotating the lid relative to the fluid collector while applying a downward force to engage a plurality of tabs included on the fluid collector in a corresponding plurality of acceptors included on the lid, wherein the airtight seal is formed by a first gasket included in the lid that interfaces with a first mating surface of the fluid collector; and applying negative pressure further comprises: supplementing the airtight seal by drawing the lid onto the fluid collector via the negative pressure to engage a second gasket included in the fluid collector that interfaces with a second mating surface of the lid.

In some embodiments, the method further comprises, before securing the solids filter into the fluid containment region through the lid: assembling the solids filter from a first section and a second section, wherein the first section is selected based on a first number, first location, and first bore diameter of a first plurality of fluid outlets included in the first section and the second section is selected based on a second number, second location, and second bore diameter of a second plurality of fluid outlets included in the second section.

### BRIEF DESCRIPTION OF THE DRAWINGS

The accompanying figures depict various elements of the one or more embodiments of the present disclosure, and are not considered limiting of the scope of the present disclosure.

In the Figures, some elements may be shown not to scale with other elements so as to more clearly show the details. Additionally, like reference numbers are used, where possible, to indicate like elements throughout the several Figures.

It is contemplated that elements and features of one embodiment may be beneficially incorporated in the other embodiments without further recitation or illustration. For example, as the Figures may show alternative views and time periods, various elements shown in a first Figure may be omitted from the illustration shown in a second Figure without disclaiming the inclusion of those elements in the embodiments illustrated or discussed in relation to the second Figure.

FIG. 1 illustrates an isometric view of the assembled collection device, according to embodiments of the present disclosure.

FIG. 2 illustrates an isometric exploded view of the collection device, according to embodiments of the present disclosure.

FIG. 3 illustrates an isometric exploded view of the solids filter, according to embodiments of the present disclosure.

FIGS. 4A-4D illustrate isometric views of one section of the solids filter and illustrate the fluid outlets defined therein, according to embodiments of the present disclosure.

FIGS. 5A-5D illustrate views of fluid flow through the solids filter, according to embodiments of the present disclosure.

FIGS. 6A and 6B illustrate isometric views of the lid interfacing with the solids filter, according to embodiments of the present disclosure.

FIG. 7 illustrates an isometric exploded view of assembly for the gaskets included in the collection device, according to embodiments of the present disclosure.

FIG. 8 illustrates as isometric view for affixing the lid to the fluid collector, according to embodiments of the present disclosure.

FIG. 9 illustrates a time lapse for twist-lock engagement between the lid and the fluid collector, according to embodiments of the present disclosure.

FIGS. 10A and 10B illustrate a time lapse for the gaskets sealing the collection device, according to embodiments of the present disclosure.

FIGS. 11A and 11B illustrate cutaway views of the collection device that include a pressure film within the fluid containment region, according to embodiments of the present disclosure.

FIG. 12 is a flowchart of a method for assembling and using a collection device, according to embodiments of the present disclosure.

### DETAILED DESCRIPTION

The present disclosure is generally related to an improved suction and aspiration collection device. The improvements described herein provide various benefits, including, but not limited to: easier use of the collection device, easier analysis of fluids and solids collected by the collection device, more robust and reliable sealing of the collection device, greater pressure differential tolerances for the collection device, and improved or simplified manufacturing techniques therefore.

The collection device may include various components that are made of translucent rigid materials, which allows for a user to view various solids collected by a solids filter during operation, presents an unobscured view of the flow of fluid through the solids filter, permits various elements to be presented at known and consistent positions within the collection device, and eases inspection of the collection device for proper assembly, material soundness, and unintentional changes in pressure.

The collection device may also include a two-gasket sealing design, which works in tandem with an improved twist-lock securing mechanism to reduce the force needed to manually seal the collection device, while ensuring that the collection device remains sealed with greater force to maintain the seal with greater pressure differentials and over longer periods of time when active suction is removed or paused.

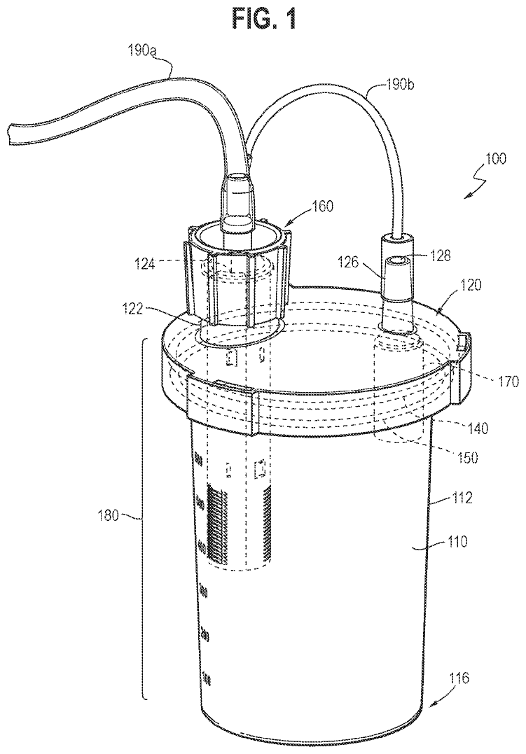

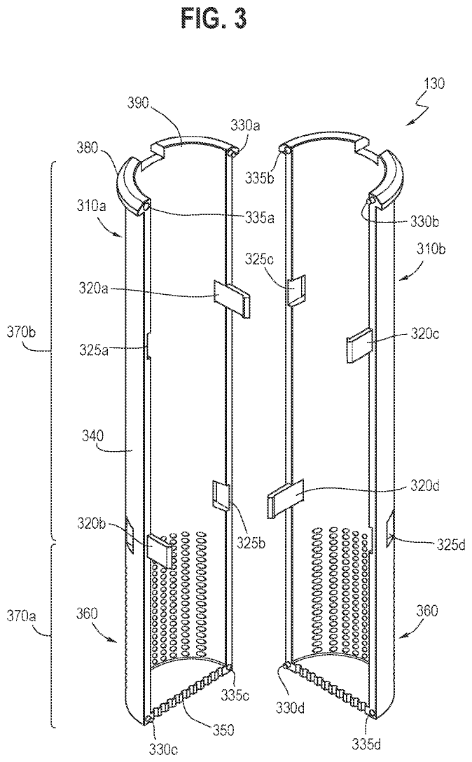

FIG. 1 illustrates an isometric view of the collection device 100 when assembled, according to embodiments of the present disclosure. The collection device 100 includes a fluid collector 110 that is covered by a lid 120 though which a solids filter 130 is secured by a cap 160 (shown in FIGS. 3, 5C-5D, and 6A-6B), and to which a return filter 170, and various tubing 190a-b are attached to draw fluid into the collection device via an imparted vacuum.

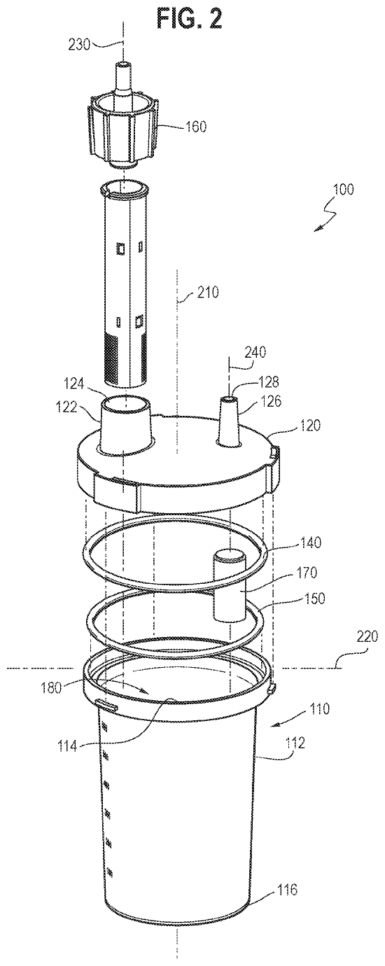

The fluid collector 110 includes a wall 112 that is joined on a first side to a base 116 and defines, on a second side (opposite to the first side) an opening 114 (shown in FIG. 2) that the lid 120 seals when engaged to the fluid collector 110. Although illustrated in FIG. 1 with a substantially cylindrical form factor, in various embodiments, the fluid collector 110 may take other shapes including, but not limited to: cones, pyramids, other shapes, and a combinations thereof (e.g., similar to Erlenmeyer flasks), and combinations thereof. In some such embodiments, the wall 112 may encompass or otherwise reduce or omit the base 116. In some such embodiments, if the base 116 is omitted, the fluid collector 110 may be held upright by a support structure (e.g., supported by a ring stand, where the conical wall 112 is lowered into the ring until the walls 112 contact the ring support structure to hold the fluid collector 110 in place). In various embodiments, the fluid collector 110 is made of a translucent material such as but not limited to plastic or glass.

As used herein, the term “translucent” is intended to include the term “transparent” such that both describe materials that light (in at least one band of the visible or near visible wavelengths) is permitted to substantially pass through the material, with or without scattering due to internal or external interfaces. Accordingly, when an element is described herein as translucent, a person (such as a physician, operator, or user) is able to see through the associated material. In various embodiments, a material that is translucent allows between X and 100 percent of light to pass through the material (with the remainder being absorbed or reflected), where X may be any number that allows a person to see through the translucent material, for example, between 50 and 99. In various embodiments, the wavelength of light that the material is judged to be translucent to may include light between at least one of the bands of approximately: 310-1100 nanometers (nm), 380-750 nm, 700-600 nm, 600-580 nm, 580-550 nm, 550-475 nm, 475-450 nm, 450-400 nm, and combinations thereof.

The lid 120 is configured to selectively interface with the fluid collector 110 and seal the opening 114 to define a fluid containment region 180 to collect various fluids within. To introduce the fluid into the fluid containment region 180, the lid 120 includes am intake port 122 that defines a first through-hole 124 through the lid 120, that is substantially parallel to the plane in which the opening 114 is defined. To apply suction (via vacuum or other negative pressure) to the fluid containment region 180 and draw the fluid in through the first through-hole 124, the lid 120 includes a suction port 126 that defines a second through-hole 128 through the lid 120, which may also be substantially parallel to the plane in which the opening 114 is defined. In various embodiments, the lid 120 is made of a translucent plastic or glass, which may be the same material as or a different material from the fluid collector 110, but may also be made of an opaque material in other embodiments.

The intake port 122 is configured to hold a solids filter 130 in place through the first through-hole 124 and into the fluid containment region 180. A cap 160 is configured to engage with the intake port 122 to hold the solids filter 130 in place, and to hold intake tubing 190a in place to connect a fluid source (e.g., a vascular access device, an aspiration catheter, a stent, a micro-catheter, a port, or other patient interface device) to the collection device 100. In various embodiments, the cap 160 is held in place over the solids filter 130 via a pressure seal, but may also be held in place via adhesives, twist-lock mechanisms, combinations thereof and other attachment mechanisms. In various embodiments, the intake port 122 includes a one-way valve or adjustable valve to allow an external pump or vacuum source (not illustrated) to apply suction to the fluid containment region 180, and to seal and maintain pressure in the fluid containment region 180 when suction is no longer applied.

The suction port 126 is configured to hold vacuum tubing 190b in place to connect an external pump or vacuum source (not illustrated) to the collection device 100. In various embodiments, the suction port 126 includes a one-way valve to allow the external pump or vacuum source (not illustrated) to apply suction to the fluid containment region 180 when active and connected via the vacuum tubing 190b, and to seal the second through-hole 128 when suction is no longer applied. Additionally, a return filter 170 may be connected to the second through-hole 128 within the fluid containment region 180 to reduce the amount or likelihood of fluids (and any contaminants therein) being drawn into the vacuum tubing 190b. In various embodiments, the return filter 170 is made of a foam or other porous material configured to allow the movement of air out of the fluid containment region 180 via the vacuum tubing 190b, but impede a bodily fluid or aerosolized contaminant (e.g., bacteria, viruses) from entering the vacuum tubing 190b.

During operation of the collection device 100, a filtration target (e.g., a blood vessel) is placed in fluid communication with a vacuum source via the intake tubing 190a and the vacuum tubing 190b so that negative pressure exerted on a fluid draws the fluid and any solids carried therein via the intake tubing 190a into the fluid containment region 180 through the solids filter 130. The solids filter 130 (shown in detail in FIGS. 3-5D) is configured to be selectively interfaced with the intake port 122 and to be inserted through the first through-hole 124 at a predefined depth into the fluid containment region 180. The solids filter 130 includes a plurality of fluid outlets that are located at a distal end relative to where the solids filter 130 interfaces with the intake port 122. In various embodiments, the solids filter 130 may be uninstalled or de-interfaced from the intake port 122 (after removing the cap 160) to allow for the retrieval or closer analysis of any solids captured from the fluid flowing into the fluid containment region 180 via the intake tubing 190a and the solids filter 130.

In various embodiments, the solids filter 130 is made of a translucent plastic or glass, which may be the same material as or a different material from, that used to construct the fluid collector 110. According, a user can visually detect whether the solids filter 130 has collected any solids, while the solids filter 130 is installed in the fluid containment region 180 and while the collection device 100 is actively receiving fluids. The ability to visual detect solids improves the usability and functionality of the collection device 100 because it allows a user (e.g., physician, operator) to visually inspect the solids filter 130 for any collected contents without releasing the vacuum or removing the lid 120, thereby maintaining negative pressure within the fluid containment region 180, maintaining a sterile environment, and reducing procedure time. Unlike opaque filters or filters with reduced visibility that require a user pause the procedure to manually inspect the filter for solid contents, a translucent or transparent solids filter 130 may allow a user to visually assess the contents mid-procedure without releasing the vacuum or taking the collection device 100 apart. Additionally, by constructing the solids filter 130 out of a rigid material, rather than a textile or mesh, any solids collected by the solids filter 130 will be held at a consistent height within the solids filter 130, and will not distort the shape of the solids filter 130 during continued use. Example features of the solids filter 130 are discussed in greater detail in regards to FIGS. 3-6.

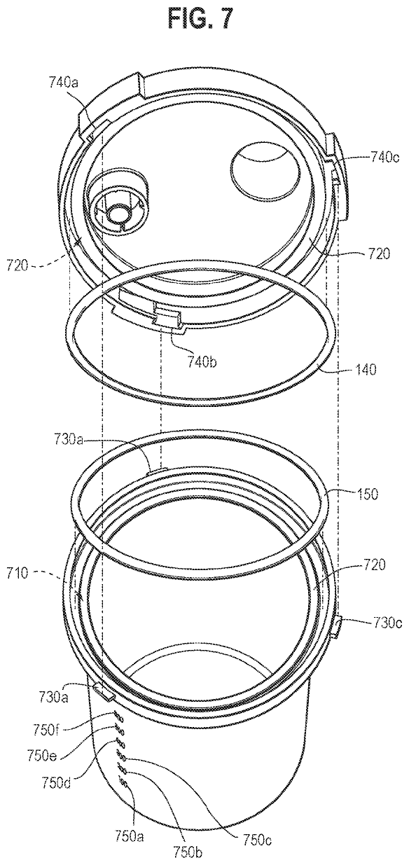

To ensure that the fluid collector 110 and the lid 120 seal tightly, and maintain the seal once formed, a first gasket 140 and second gasket 150 are positioned between the fluid collector 110 and the lid 120. Each of the first gasket 140 and the second gasket 150 are made of a compressible material, such as a rubber or rubberized plastic, to deform and ensure tight contact between two surfaces when pressure is applied to the first gasket 140 and the second gasket 150. Additional gaskets (not shown) may also be used to provide additional sealing properties. Multiple gaskets (such as the first gasket 140 and the second gasket 150) allow the collection device 100 to maintain a vacuum seal for longer durations, at higher pressure differentials, and within challenging environments, and may reduce the likelihood that the user needs to spend time during a procedure to troubleshoot issues such as air leaks or inconsistent pressures. In various embodiments, the pressure may be maintained on the first gasket 140 and the second gasket 150 via various locking or connecting mechanisms included in the fluid collector 110 and lid 120. Example features of how the fluid collector 110 and the lid 120 are interfaced with one another and the seal is maintained by the first gasket 140 and the second gasket 150 are discussed in greater detail in regard to FIGS. 7-10B.

FIG. 2 illustrates an isometric exploded view of the collection device 100, according to embodiments of the present disclosure. The various elements of the collection device 100 are assembled relative to a central axis 210 and an opening plane 220 defined by the opening 114 in the fluid collector 110 that the lid 120 seals. Both the fluid collector 110 and the lid 120 are substantially circular in cross-sectional area (at least at the areas used to mate the two components with one another) so that the lid 120 may seal the opening 114 by rotating relative to the fluid collector 110 about the central axis 210 to engage various securing features, such as the twist-lock mechanisms and paired gaskets/mating surfaces discussed in greater detail in regard to FIGS. 7-10B.

Although the collection device 100 may be held in various orientations, for ease of understanding, the present disclosure uses relative positional terms (e.g., upward, above, downward, below, higher, lower, top, bottom) in a direction oriented along the central axis 210 (e.g., the Z direction) using the base 116 of the fluid collector 110 as a reference point, where elements that are closer to the base 116 are considered “below” elements that are further from the base 116 and elements that are further from the base 116 are considered “above” elements that are closer to the base 116. Similarly, unless stated otherwise, rotation of an element of the collection device 100 relative to another element of the collection device 100 shall be understood to be rotation substantially about the central axis 210. Additionally, unless stated otherwise, relative terms such as “inner” and “outer” shall be understood in relation to the central axis 210, where elements that are closer to the central axis 210 are considered “inner” elements relative to elements further from the central axis 210 and elements that are further from the central axis 210 are considered “outer” elements relative to elements that are closer to the central axis 210.

In various embodiments, the substantially circular cross-sectional areas may be tapered relative to the opening plane 220, such that one or more of the fluid collector 110 and lid 120 taper or flare with increased distanced from the opening plane 220 to have cross-sectional areas that differ over the height of the respective component.

Although the various securing features are generally arranged concentrically or with radial symmetry from the central axis 210, various elements may be offset or otherwise disposed off-axis relative to the central axis 210. For example, the intake port 122 defined in the lid 120 that defines the intake through-hole 124 by which the solids filter is 130 disposed into the fluid containment region 180 may be defined at a first distance from the central axis 210 on an intake axis 230 substantially parallel to the central axis 210. Similarly, the suction port 126 defined in the lid 120 that defines the suction through-hole 128 by which suction or negative pressure is applied to the fluid containment region 180 may be defined at a second distance from the central axis 210 on a substantially parallel suction axis 240.

In various embodiments, the first and second distances (from the central axis 210) are equal, such that the intake axis 230 and the suction axis 240 are defined at a shared radius from the central axis 210. In other embodiments, the first and second distances are un-equal, which may improve the stability of the assembled collection device 100 to account for the greater weight of the cap 160 and solids filter 130 compared to the return filter 170 and different forces applied by fluids entering via the intake port 122 and air being evacuated by the suction port 126.

In various embodiments, by placing the intake port 122 off-axis relative to the central axis 210, the solids filter 130 is disposed closer to one section of the wall 112 of the fluid collector 110, thereby improving visibility when viewing the solids filter 130 (and any captured solids therein) from that section of the wall 112 compared to a solids filter 130 aligned concentrically with the central axis 210.

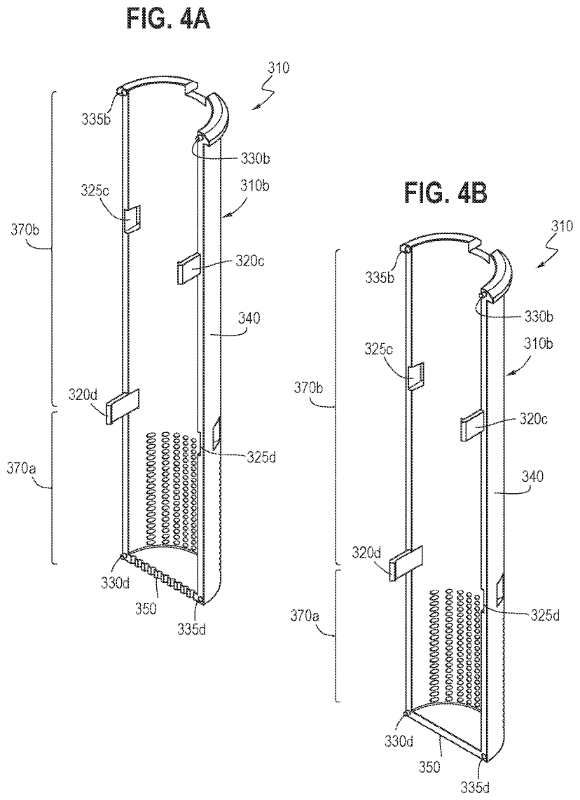

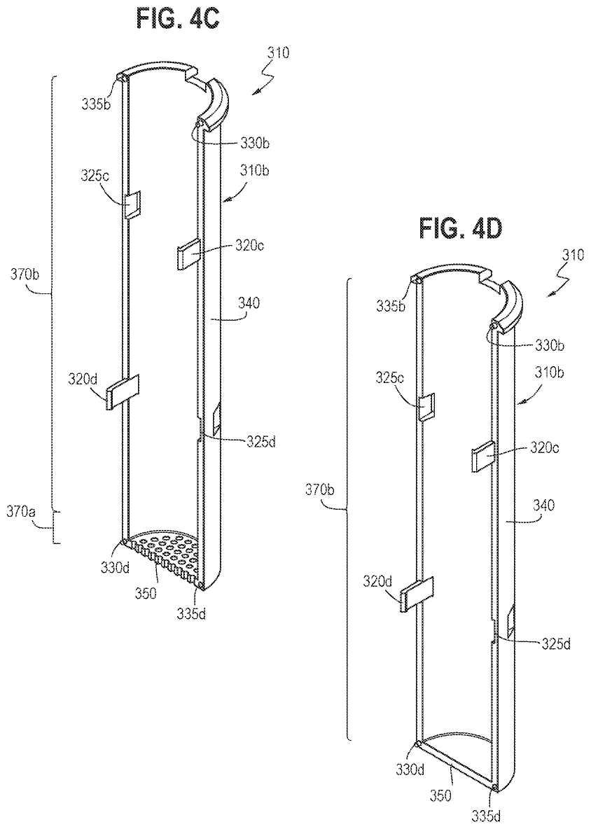

FIG. 3 illustrates an isometric exploded view of the solids filter 130, according to embodiments of the present disclosure. In various embodiments, the solids filter 130 is made of two sections 310a-b (generally or collectively, section 310) that mate together via a plurality of locking tabs 320a-d (generally or collectively, locking tabs 320) corresponding to a plurality of locking slots 325a-d (generally or collectively, locking slots 325) and a plurality of alignment tabs 330a-d (generally or collectively, alignment tabs 330) corresponding to a plurality of alignment slots 335a-d (generally or collectively, alignment slots 335 325). In various embodiments, the mating surfaces between the two sections 310a-b can be further reinforced with an adhesive, resin, or the like to supplement locking tabs/slots 320/325 and the alignment tabs/slots 330/335 and provide a tighter seal between the two sections 310a-b. Although the two sections 310a-b are illustrated as being identical, in various embodiments, an operator or designer can select sections 310 with different layouts of fluid outlets 360 (e.g., such as those discussed in relations to FIGS. 4A-4D) to affect the fluid flow when the collection device 100 is in operation.

In various embodiments, it is advantageous for the two sections 310a-b to be identical for ease of manufacturing, reduction of dies, and minimizing the number of unique components to simplify assembly and ease the replacement of any lost, missing, or mis-sized components.

In various embodiments, the locking tabs 320 include a distal end with an oversized chamfered tip configured to pull the two sections 310 into contact with one another by engaging with the portions of the other section 310 that define the corresponding locking slots 325 to define cantilevered snap-joints.

In various embodiments, the alignments tabs 330 include protrusions that are configured to fit within depressions defined in the material of the other sections 310 by the alignment slots 335, to engage the two sections 310 via pressure and friction or to help prevent torsion from misaligning the sections 310.

Each section 310 defines half of the directing face 340 of the solids filter 130, and a half of the collecting face 350 of the solids filter 130. In various embodiments, one or both of the directing face 340 and the collecting face 350 include a plurality of fluid outlets 360, which are discussed in greater detail in regards to FIGS. 4A-4D.

In embodiments that define fluid outlets 360 through the directing face 340, the directing face 340 can be understood to have two portions 370a-b based on where the fluid outlets 360 are defined. When viewed from the side of the directing face 340 connected to the collecting face 350, a first portion 370a of the directing face 340 includes the fluid outlets 360, and at a height where no further fluid outlets 360 are defined, a second portion 370b is defined. Stated differently, the first portion 370a is defined closer to the collecting face 350 and further from an intake opening 390 (relative to the second portion 370b) and includes the plurality of fluid outlets 360 defined in the directing face 340 and any area of the directing face 340 proximal to the collecting face 350 relative to a fluid outlet 360 defined in the directing face 340. Similarly, the second portion 370b (having to fluid outlets 360) is defined further from the collecting face 350 and closer to an intake opening 390 (relative to the first portion 370a) and includes any area of the directing face 340 proximal to the intake opening 390 relative to a fluid outlet 360 defined in the directing face 340. In another embodiment, the plurality of fluid outlets 360 may be disposed along the entire length of directing face(s) 340, effectively minimizing or eliminating the second portion 370b. In a further embodiment (as shown in FIG. 4C), the plurality of fluid outlets 360 may be disposed only in the collecting face 350, effectively eliminating the first portion 370a.

In various embodiments, the numbers, orientations, and dimensions of the fluid outlets 360 may vary based on the intended flowrate of the fluid through the solids filter 130, expected viscosity of the fluid, desired size of particulates or solids to be collected, and combinations thereof. Similarly, the percentages of the directing face 340 defined as a first portion 370a or a second portion 370b may vary in different embodiments. By specifying where the first portion begins (or the second portion ends) relative to the intake opening 390, a designer can control where the fluid enters the fluid containment region 180 and where any collected solids are held. Additionally, by using a rigid (and translucent) material to contract the solids filter 130, the designer ensures that any collected solids are visible at a consistent location in the solids filter 130, unlike textile or mesh filters, which may deform when in use or when solids are present.

As illustrated, the locking tabs 320 and locking slots 325 are defined exclusively in the second portion 370b, while the alignment tabs 330 and alignment slots 335 are defined in both the first portion 370a and second portion 370b. In various embodiments, however, a designer may incorporate locking tabs 320 and locking slots 325 in the first portion 370a.

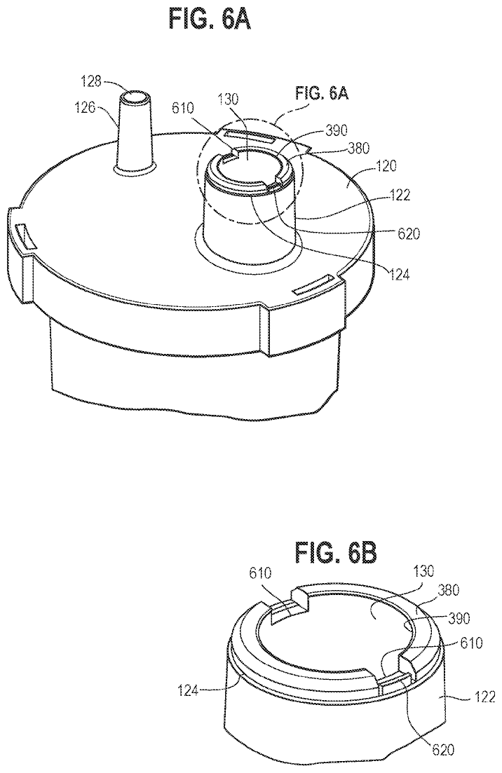

During operation of the collection device 100, the fluid (and any solids carried therewith) enters the solids filter 130 through an intake opening 390 defined at an opposite end of the directing face 340 relative to the collecting face 350. As illustrated, the translucent walls (e.g., the directing faces 340) of the two mated sections 310 describe an open cylindrical prism where the intake opening 390 defines a plane substantially parallel to the collecting face 350 that is bounded by a lip 380. As described in greater detail in regard to FIGS. 6A and 6B, the lip 380 is a greater circumference than the directing face 340 or the collecting face 350, and may include various alignment features configured to engage with the lid 120 to orient the solids filter 130.

Although illustrated as substantially circular in cross-sectional area, the solids filter 130 may be ovoid or another shape, which may act as an alignment feature using the relative shapes of the solids filter 130 and intake port 122. Additionally, although illustrated as substantially circular in area, the collecting face 350 may be hemispherical, conic, or another shape in various embodiments. Additionally or alternatively, the directing faces 340, when defining a conic solids filter 130, may encompass or otherwise reduce or omit the collecting face 350.

FIGS. 4A-4D illustrate isometric views of one section 310 of the solids filter 130 to detail the fluid outlets 360 defined therein, according to embodiments of the present disclosure. FIG. 4A illustrates that the fluid outlets 360 can be defined in both the directing face 340 and the collecting face 350, allowing the solids filter 130 to output fluid from the sides and bottom thereof during operation. FIG. 4B illustrates that the fluid outlets 360 can be defined exclusively in the directing face 340, allowing the solids filter 130 to output fluid from the sides thereof during operation. FIG. 4C illustrates that the fluid outlets 360 can be defined exclusively in the collecting face 350, allowing the solids filter 130 to output fluid from the bottom thereof during operation (allowing all of the directing face 340 thereof to be considered a second portion 370b). FIG. 4D illustrates that the fluid outlets 360 can omitted in one section 310 that is intended to be mated with another section 310 that does include fluid outlets 360, thereby directing fluid outflow through one half of the solids filter 130, and the operator with an improved view within the solids filter 130 (e.g., unimpeded by refraction index differences due to the fluid outlets 360) or the ability to remove the solids filter 130 at an orientation to avoid or reduce leakage of any fluids still within the solids filter 130.

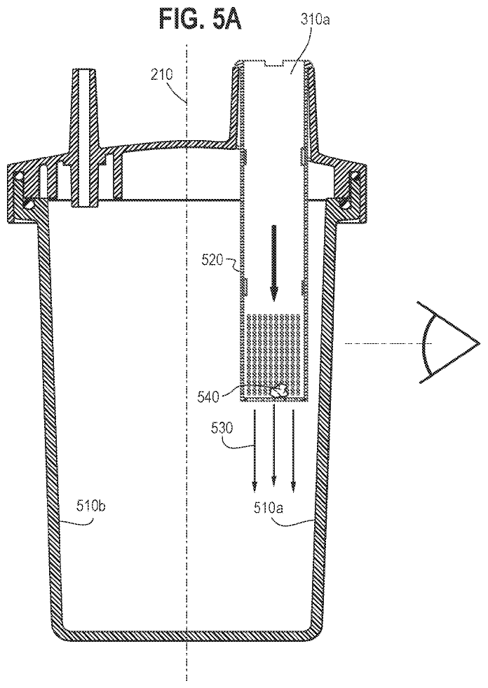

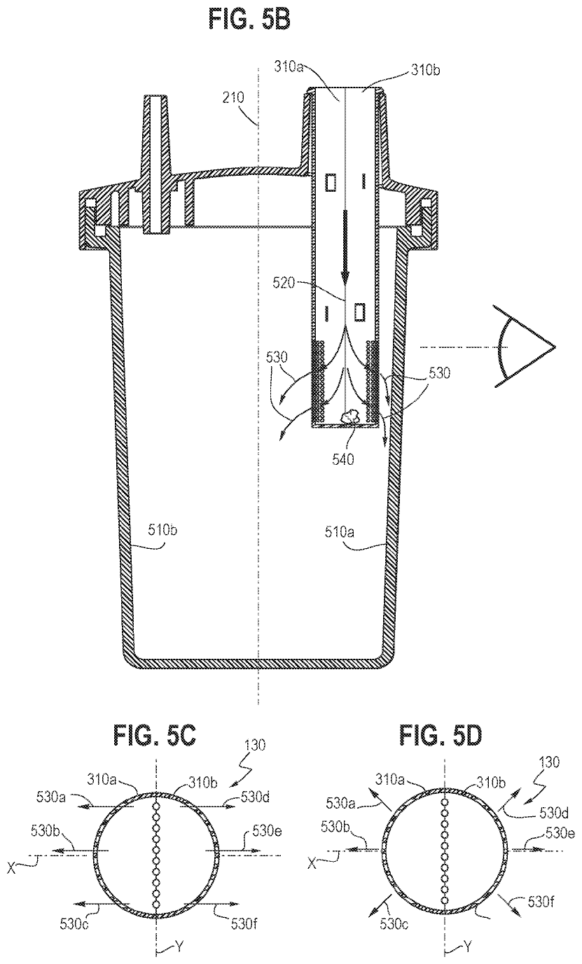

FIGS. 5A-5D illustrate views of fluid flow through the solids filter 130, according to embodiments of the present disclosure. As illustrated, when a solids filter 130 is installed in an operational collection device 100, fluid 530 enters the solids filter 130 via the intake opening 390, and exits (into the fluid containment region 180) via the fluid outlets 360. Depending on the number, presence, and orientation of the fluid outlets 360, and the flow rate of the fluid 530 into the solids filter 130, the fluid 530 may be directed in various directions to improve the ability of a viewer to observe various solids 540 captured in the solids filter 130.

As shown in the cutaway views in FIGS. 5A and 5B, the non-concentric placement of the solids filter 130 (relative to the central axis 210) places the solids filter 130 closer to one portion of the wall 112 of the fluid collector 110, which may be designated as the viewing wall 510a, and further from other portions of the wall 112, which may be designated as the distal wall 510b.

Although in the cutaway views illustrated in FIGS. 5A and 5B show one portion of the viewing wall 510a, the viewing wall 510a may include various portions of the wall 112 that are within a designated proximity to the solids filter 130. When one or more of the fluid collector 110 and the solids filter 130 describe generally circular, conic, truncated-conic, or other prisms having one or more radii, the viewing wall 510a may be defined as the internal point (or line) on the inner portion of the wall 112 closest to the outer portion of the solids filter 130, and the area of the wall 112 within a given arc distance of the closest point (or line). For example, the viewing wall 510a may include a section of the wall 112 the defines an arc of 30 degrees, 60 degrees, 90 degrees, 120 degrees, or other designated angle centered on the closest point (or line) of the wall 112 to the solids filter 130. Additionally or alternatively, the viewing wall 510a may describe those portions of the wall 112 within a threshold distance of any portion of the solids filter 130 (e.g., within X centimeters (cm))

Although in the cutaway views illustrated in FIGS. 5A and 5B show the portion of the distal wall 510b opposite to the illustrated portion of the viewing wall 510a, any portion of the wall 112 not designated as the viewing wall 510a may be described as the distal wall 510b.

FIG. 5A illustrates a cutaway side view (e.g., in a ZX plane) where a solids filter 130 disposed in the fluid containment region 180 of a collection device 100 is oriented to direct the fluid 530 through those fluid outlets 360 defined in the directing faces 430 away from the viewing wall 510a (e.g., into or out of the page in the Y direction). When using a two-section construction (see e.g., FIG. 3) for the solids filter 130, the mating line 520 between the two sections 310a-b may be presented towards the closest point (or line) between the wall 112 and the solids filter 130 to direct fluid 530 substantially perpendicularly through the fluid outlets 360 defined in directing face 340 the viewing plane.

Due to the orientation shown in FIG. 5A, a first section 310a is visible, which a second section 310b (not illustrated) is obscured by the first section 310a; however, both the first section 310a and second section 310b are visible from the viewing plane through the viewing wall 510a. Accordingly, the mating line 520 between the first section 310a and the second section 310b is visible to the viewer.

In various embodiments, the operator may direct the fluid 530 substantially evenly relative to the viewing plane (e.g., both into and out of the page) by using identical sections 310 with fluid outlets 360 defined in the directing faces 340 (see e.g., FIG. 4A). In other embodiments, the operator may preferentially direct the fluid 530 in one direction relative to the viewing plane (e.g., more into or more out of the page) by using sections 310 with different numbers, locations, bore diameters of fluid outlets 360 relative to one another. For example, the operator may use one section 310 that includes fluid outlets 360 in the directing face 340 (see e.g., FIG. 4A-4B) and one section that excludes fluid outlets 360 in the directing face 340 (see e.g., FIG. 4C-4D) to direct the fluid 530 in one direction and not the other (e.g., only into or only out of the page).

Although FIG. 5A illustrates the fluid 530 exiting the solids filter 130 through the directing face 340 and the collecting face 350, in various embodiments, an operator can select the sections 310 of the solids filter 130 to include or exclude fluid outlets 360 in the respective directing face 340 or collecting face 350. Accordingly, the fluid 530 may flow through both the directing face 340 and collecting face 350, exclusively through the directing face 340 (e.g. when omitting fluid outlets 360 in the collecting face 350), or exclusively through the collecting face 350 (e.g. omitting fluid outlets 360 in the directing face 340). Additionally or alternatively, depending on flow rate of the fluid 530 into and out of the solids filter 130, and the relative height of the first fluid outlets 360 defined in the directing face 340 (e.g., proximity to the collecting face 350), the solids filter 130 may initially output the fluid 530 through the collecting face 350 until the level of the fluid 530 in the solids filter 130 reaches the first fluid outlets 360 defined in the directing face 340.

FIG. 5B illustrates where a solids filter 130 disposed in the fluid containment region 180 of a collection device 100 is oriented to direct the fluid 530 through those fluid outlets 360 defined in the directing faces 340 towards the illustrated portions of the viewing wall 510a (e.g., in the X direction) and the distal wall 510b. When using a two-section construction (see e.g., FIG. 3) for the solids filter 130, the mating line between the two sections 310a-b may be presented away from the closest point (or line) between the wall 112 and the solids filter 130 to direct fluid 530 substantially perpendicularly through the fluid outlets 360 defined in directing face 340 towards the illustrated portions of the viewing wall 510a and distal wall 510b.

Due to the orientation shown in FIG. 5B, a first section 310a is visible from the viewing wall 510a, but the second section 310b may obscured by the first section 310a. Accordingly, the mating line 520 between the first section 310a and the second section 310b, while visible in FIG. 5B, may be obscured from the viewer.

In various embodiments, the operator may direct the fluid 530 substantially evenly relative to the viewing plane (e.g., both towards and away from the viewing wall 510a) by using identical sections 310 with fluid outlets 360 defined in the directing faces 340 (see e.g., FIG. 4A). In other embodiments, the operator may preferentially direct the fluid 530 in one direction towards or away from the viewing plane by using sections 310 with different numbers, locations, bore diameters of fluid outlets 360 relative to one another. For example, the operator may use one section 310 that includes fluid outlets 360 in the directing face 340 (see e.g., FIG. 4A-4B) and one section that excludes fluid outlets 360 in the directing face 340 (see e.g., FIG. 4C-4D) to direct the fluid 530 in one direction and not the other (e.g., only towards or away from the viewing wall 510a).

Although FIG. 5B illustrates the fluid 530 exiting the solids filter 130 through the directing face 340 and the collecting face 350, in various embodiments, an operator can select the sections 310 of the solids filter 130 to include or exclude fluid outlets 360 in the respective directing face 340 or collecting face 350. Accordingly, the fluid 530 may flow through both the directing face 340 and collecting face 350, exclusively through the directing face 340 (e.g. when omitting fluid outlets 360 in the collecting face 350), or exclusively through the collecting face 350 (e.g. omitting fluid outlets 360 in the directing face 340). Additionally or alternatively, depending on flow rate of the fluid 530 into and out of the solids filter 130, and the relative height of the first fluid outlets 360 defined in the directing face 340 (e.g., proximity to the collecting face 350), the solids filter 130 may initially output the fluid 530 through the collecting face 350 until the level of the fluid 530 in the solids filter 130 reaches the first fluid outlets 360 defined in the directing face 340.

FIG. 5C illustrates, in an XY plane substantially parallel to the intake opening 390 and collecting face 350, a first orientation of the fluid outlets 360 in the directing faces 340 that are substantially parallel to one each other. FIG. 5D illustrates a second orientation of the fluid outlets 360 in the directing face 340 that are radially oriented relative to the directing face 340. In various embodiments, a directing face 340 may include parallel, radial, or both radial and parallel (at different heights in the Z direction) orientations for the flow-paths for fluid 530 through the fluid outlets 360. The various fluid outlets 360 may be formed as voids in an injection molding process (e.g., where a mold prevents flow of material into an area to define a fluid outlet 360), drilled or etched (via chemical or mechanical means) through the material of the section 310, or as areas in which material is not deposited in a three-dimensional printing or additive manufacturing process. Accordingly, the size, shape, bore diameter, orientation, and pattern of the various fluid outlets 360 may be selected based on the flow characteristics desired, and the manufacturing technique used to produce the sections 310.

FIGS. 6A and 6B illustrate isometric views of the lid 120 interfacing with the solids filter 130, with 6B providing a detailed view of the intake port 122 shown in FIG. 6A, according to embodiments of the present disclosure. As illustrated, the lip 380 of the solids filter 130 has a greater circumference from the rest of the solids filter 130, which engages with the intake port 122 to position the solids filter 130 through the first through-hole 124. In various embodiments, the lip 380 may define one or more filter alignment features 610 to engage with corresponding port alignment features 620 defined in the intake port 122. The filter alignment features 610 may include cut-outs from the lip 380 that are configured to interface with extensions from the intake port 122 that define the port alignment features 620.

For example, the filter alignment features 610 may define an area of reduced thickness relative to the rest of the lip 380 that cantilevered clips (that form the corresponding port alignment features 620) engage with the filter alignment feature 610 to form a snap-joint to align and hold the solids filter 130 in place relative to a viewing wall 510a (e.g., per FIG. 5A or 5B). In various embodiments, the relative thickness of the lip 380 prevents the port alignment features 620 from engaging with the solids filter 130 at sections other than the reduced-thickness filter alignment features 610.

Similarly, positively defined protrusions (e.g., pegs or tabs) from the intake port 122 may engage with through-holes or cut-outs in the lip 380 to ensure a desired orientation of the solids filter 130 through the intake port 122. In other embodiments, negatively defined cutouts or inclusions in the intake port 122 may engages with positively defined protrusions from the lip 380 to ensure a desired orientation of the solids filter 130 through the intake port 122. Additionally or alternatively, when the intake port 122 and the solids filter 130 are defined to have non-circular cross-sections (e.g., ovular, flatten circles, rectangular, etc.), the corresponding shapes for the solids filter 130 and the intake port 122 can ensure a desired orientation of the solids filter 130 through the intake port 122.

FIG. 7 illustrates an isometric view of assembly for the gaskets included in the collection device 100, according to embodiments of the present disclosure. The lid 120 includes a first compressible sealing gasket 140 that is oriented concentrically with the lid 120 relative to the central axis 210 and the fluid collector 110 includes a second compressible sealing gasket 150 that is also oriented concentrically with the fluid collector 110 relative to the central axis 210. When engaging the lid 120 with the fluid collector 110, each orients the respective gasket with the other so that a first mating surface 710 defined on the fluid collector 110 is oriented to interface with the first gasket 140, and a second mating surface 720 defines on the lid 120 is oriented to interface with the second gasket 150.

Each of the paired first mating surface 710/gasket 140 and second mating surface 720/gasket 150 are configured to engage one another when the lid 120 is affixed to the fluid collector 110 so that pressure between the lid 120 and the fluid collector 110 compresses the gaskets, and forms a seal between the fluid containment region 180 and external environment. To ensure the pressure is sufficient to maintain the seal, the lid 120 includes a plurality of acceptors 740a-c (generally or collectively, acceptors 740) and the fluid collector 110 include a corresponding plurality of tabs 730a-c (generally or collectively, tabs 730) to form twist-lock joints, which are discussed in greater detail in regard to FIG. 9. Generally, the tabs 730 are male twist-lock engagement features while the acceptors 740 are female twist-lock engagement features that form twist-lock pairs when engaged with one another.

Although three twist-lock pairs of acceptors 740 and tabs 730 are shown in FIG. 7, in various embodiments, more or fewer twist-lock pairs may be included. In some embodiments, the various twist-lock pairs are disposed substantially evenly around the circumference of the collection device 100 so that the illustrated three twist-lock pairs are located approximately 120 degrees away from one another, but with N pairs, each twist-lock pair may be located approximate 360/N degrees away from each neighboring twist-lock pair so that each is positioned at a given arc distance clockwise from a first neighbor and the same given arc distance counterclockwise from a second neighbor.

In various embodiments, one twist-lock pair of acceptors 740 and tabs 730 includes a tab 730 that is larger than the other tabs 730 and that can only be accepted by one acceptor (e.g., due to being is too large to be accepted by other acceptors 740 or having a different shape) to thereby ensure a specified orientation of the lid 120 relative to the fluid collector 110. In various embodiments, each tab 730 is substantially identical to the other tabs 730 and each acceptor 740 is substantially identical to the other acceptors 740 to allow the lid 120 to selectively interface with the fluid collector 110 in various rotational alignments.

The fluid collector 110 may include various graduations 750a-e (generally or collectively, graduations 750) to indicate a fill volume collected by the fluid collector 110. In various embodiments, the graduations 750a-e may extend around the circumference of the fluid collector 110 or may be located at discrete locations around the circumference of the fluid collector 110. When located at discrete locations, the graduations 750 may be located relative where the solids filter 130 is positioned within the fluid collector 110 based on the positioning of the twist-lock pairs and the intake port 122. Although FIG. 7 illustrates five graduations 750a-e, various embodiments may include more or fewer than five graduations 750 associated with various volumes.

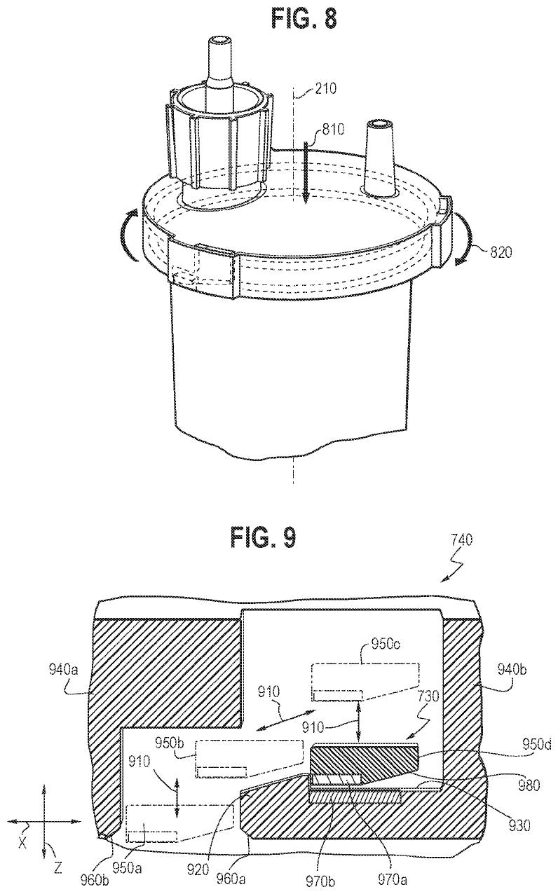

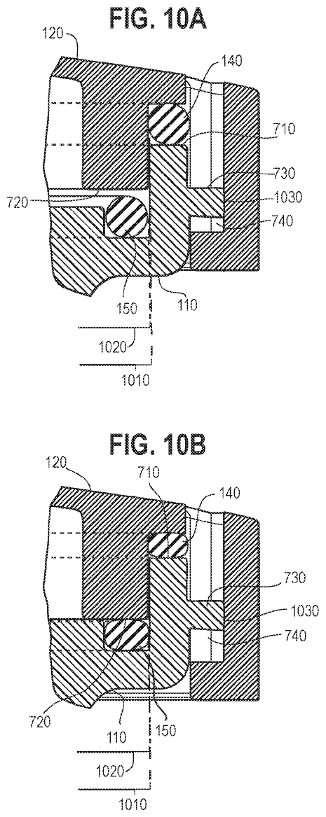

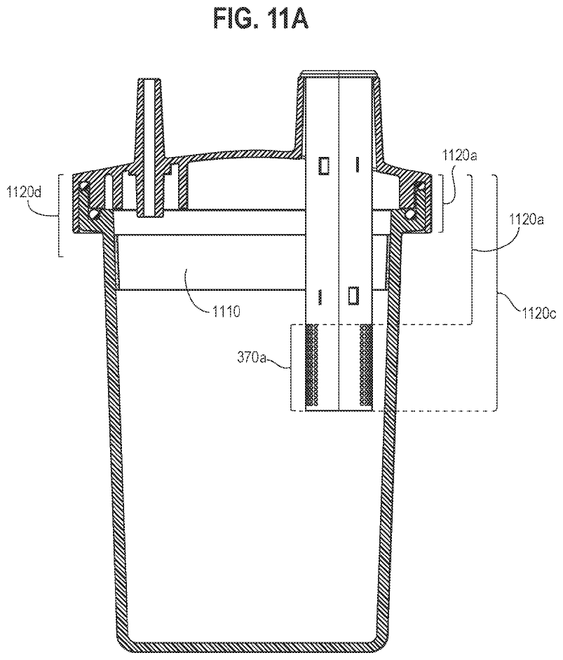

FIG. 8 illustrates as isometric view for selectively affixing the lid 120 to the fluid collector 110, according to embodiments of the present disclosure. The various engagement interfaces between the lid 120 and fluid collector 100 are also discussed in relation to FIGS. 9, 10A, and 10B. Although the provided discussion primarily focuses on placing the lid 120 in the engaged configuration with the fluid collector 110 and selectively disengaging the lid 120 from the fluid collector 110 by the user acting primarily on the lid 120 with the fluid collector 110 placed below the lid 120, the described actions may similarly be applied via a user acting primarily on the fluid collector 110 or the user acting on both the lid 120 and the fluid collector 110 in various orientations.

When attaching the lid 120 to the fluid collector 110, an operator exerts a downward force 810 to engage the tabs 730 into the acceptors 740, and then applies a rotational force 820 to the lid 120 relative to the fluid collector 110 in a first direction relative to a central axis 210 (e.g., clockwise). Internal features to the acceptor 740 (discussed in greater detail in regard to FIG. 9) prevent rotation of the lid 120 past a predefined distance relative to the fluid collector 110, and once the lid 120 reaches this predefined distance, the operator may release the downward force 810 and rotational force 820 to maintain the seal in the collection device 100.

When detaching the lid 120 from the fluid collector 110, an operator exerts a downward force 810 to disengage the tab 730 from the acceptors 740, and then applies a rotational force to the to the lid 120 relative to the fluid collector 110 in a second direction relative to a central axis 210 (e.g., counterclockwise). Internal features to the acceptor 740 (discussed in greater detail in regard to FIG. 9) prevent rotation of the lid 120 in the second direction once a seal is formed until the downward force 810 releases the tab 730 from the internal features. Once released from the internal features, the operator can apply the rotational force 820 to disengage the lid 120 from the fluid collector 110 and break the seal

FIG. 9 illustrates a time lapse for twist-lock engagement between the lid 120 and the fluid collector 110, according to embodiments of the present disclosure. As illustrated, the internal features of one acceptor 740 relative to one tab 730, where the tab 730 is shown at three different times at four different positions 950a-d relative to the internal features of the acceptor 740 to illustrate selective engagement and disengagement between a tab 730 and an acceptor 740.

The acceptor 740 defines a path 910 that the tab 730 travels over when the appropriate forces are applied to the fluid collector 110 and lid 120. In various embodiments, a pair of intakes 960a-b is located at an entry/exit portion of the path 910 that accepts or ejects the tab 730 from the acceptor 740, shown as the first position 950a. The intakes 960 help align the tab 730 into the path 910 when an operator applies a downward force 810 to begin sealing the collection device 100. The intakes 960a-b therefore define sloped (e.g., non-parallel and non-perpendicular) surfaces relative to the downward force 810 to engage with the tab 730 and push that tab 730 into alignment with the acceptor 740 when the downward force 810 is applied to the tab 730 via one of the intakes 960.

The path 910 is bounded by a first boundary wall 940a (generally or collectively boundary wall 940), a second boundary wall 940b, and a ramp 920 that leads to a catchment 930 that is designed to hold the tab 730 in place in the fourth position 950d (and thereby hold the lid 120 in place to the fluid collector 110) when rotated into an engaged position, and resist rotation out of the engaged position unless a downward force 810 is applied with counter rotation to the rotational force 820 used to engage the tab 730 in the catchment 930. The boundary walls 940 prevent the tab 730 from rotating past the endpoints of the path 910 in either rotational direction. The first boundary wall 940a prevents over-rotation in the counterclockwise direction, and the second boundary wall 940b presents over-rotation in the clockwise direction.

The ramp 920 provides an upward slope that engages with an angled portion 980 of the tab 730 when in the second position 950b (and other non-illustrated positions between the first position 950a and the third position 950c) to reduce the magnitude of the downward force 810 needed to be applied when rotating the tab 730 from the first position 950a to the fourth position 950d. The ramp 920 converts a portion of the rotational force 820 as the tab 730 moves from the first position 950a to the third position 950c to a supplemental downward force that pushes the lid 120 downward as the tab 730 moves closer to the catchment 930, thus reducing strain on the operator from applying the downward force 810 directly to the lid 120. In various embodiments, the pitch of the angled portion 980 is matched to the pitch of the ramp 920 (e.g., both are angled at X degrees relative to the X direction). The angled portion 980 may make up various portions of the length (in the X direction) of the tab 730 based on the pitch, total length of the tab 730, and height (in the Z direction) of the tab 730.

When the tab 730 is moved over the catchment 930 and into the third position 950c, the operator can release the downward force 810 and allow the resisting upward force from the compressed gaskets to push the lid 120 upward and move the tab 730 to the fourth position 950. Similarly, when disengaging the lid 120 and the fluid collector 110, the operator may work in the reverse direction: applying a downward force 810 to move the tab 730 from the fourth position 950d to the third position 950c, applying a rotational force 820 to move the tab 730 from the third position 950c to the second position 950b and then the first position 950a, after which the lid 120 may be removed from the fluid collector 110.

In various embodiments, the vertical gap (in the Z direction) between the first boundary wall 940a and the ramp 920 at the portion of the ramp 920 distal to the intakes 960 and proximal to the catchment 930 is at least as great as the height of the tab 730 (in the Z direction) to allow passage of the tab 730 through the gap. Although illustrated with the tab 730 moving while in contact with the ramp 920, in various embodiments, the vertical gap may be oversized to allow an operator (that applies a downward force 810 of sufficient magnitude) to push the tab 730 in contact with an upper portion of the first boundary wall 940a, and then apply the rotational force 820 to move the tab 730 above the endpoints of the path 910 without contacting the ramp 920.

The horizontal gap (in the X direction) between the catchment 930 and the first boundary wall 940a may be less than or equal to the length (in the X direction) of the tab 730 to force engagement with the ramp 920 when engaging or disengaging the lid 120 with the fluid collector 110. In various embodiments, the length of the catchment 930 (in the X direction) is equal to or greater than the length of the tab 730. Additionally, the depth of the catchment 930 (in the Z direction, measure from a point of contact with the ramp 920) may be less than, equal to, or greater than the height of the tab 730 to generally require greater downward forces 810 to move the tab 730 from the fourth position 950d to the third position 950c with greater depths.

In various embodiments, the tab 730 includes a first magnet 970a (generally or collectively, magnet 970) and the catchment 930 includes a second magnet 970b. The magnets 970 are permanent magnets of opposing polarities that provide an attractive force when in proximity to one another to further aid in securing the tab 730 in the catchment 930 to maintain the seal between the lid 120 and the fluid collector 110, and thereby a greater downward force 810 to remove the tab 730 from the catchment 930 and break the seal. Accordingly, the designer can specify the strength of the magnets 970 to increase the magnitude of the downward force 810 required to move the tab 730 from the fourth position 950d to the third position 950c in addition to or instead of designing any increasingly greater depth for the catchment 930. In some embodiments, one of the first magnet 970a or the second magnet 970b may be replaced with a ferromagnetic insert that, although exhibiting no magnetic strength on its own, a magnet 970 can magnetically couple to when placed in proximity to the ferromagnetic insert. In various embodiments, the first magnet 970a may be included or extend into the angled portion 980 of the tab 730, or may be included exclusively outside of the angled portion 980 (e.g., in a “flat” or non-angled portion 980).

In various embodiments, the magnets 970 provide the operator with tactile or audible feedback as the lid 120 “snaps” into place on the fluid collector 110, confirming that the components are in proper alignment and that a vacuum seal has been formed.

Although FIG. 9 illustrates a path 910 and internal elements of an acceptor 740 that uses clockwise rotation to engage with lid 120 and the fluid collector 110 (and counterclockwise rotation to disengage the lid 120 and the fluid collector 110), the illustrated elements may be mirrored about the Z axis to provide counterclockwise engagement (and clockwise disengagement).

FIGS. 10A and 10B illustrate a time lapse for the gaskets sealing the collection device 100, according to embodiments of the present disclosure. FIGS. 10A and 10B illustrate a cross-sectional view of the lid 120 engaging with the fluid collector 110 with a tab 730 engaging with a corresponding acceptor 740. The first gasket 140 is included in the lid 120 to interface with a first mating surface 710 (as part of the fluid collector 110 concentrically aligned with the first gasket 140). The second gasket 150 is included in the fluid collector 110 to interface with a second mating surface 720 (as part of the lid 120 concentrically aligned with the second gasket 150). The gaskets are configured to compress when under pressure applied between the lid 120 and the fluid collector 110, such as a downward force 810 applied by an operator when engaging the lid 120 and the fluid collector 110, as maintained by the tab 730 and acceptor 740 when engaged, by negative pressure supplied via the vacuum tubing 190b, and combinations thereof, to thereby establish and maintain an airtight seal.

In various embodiments, the gaskets may be made of various compressible materials to form an airtight seal, which may include various rubbers or rubberized plastics, and the first gasket 140 may be made of the same or a different material than the second gasket 150.

The first gasket 140 encompasses the second gasket 150, such that the second gasket 150 defines a smaller ring (as measured from the outer radius 1020 thereof) than in inner radius 1010 of the ring defined by the first gasket 140. Although the gaskets are deformable under pressure, the gaskets may have a substantially circular cross-sectional areas when uncompressed, or may have cross-sectional areas of other shapes (e.g., oval, rectangular, irregular, etc.). In various embodiments, the cross-sectional areas of each of the first gasket 140 and the second gasket 150 may be substantially equal, or may be unequal with one gasket having a cross-sectional area at least ten percent larger than the other. The gaskets may be mounted in the respective lid 120 or fluid collector 110 via adhesives, pressure mounting (e.g., via the annular shape of the gaskets interfacing with a groove or depression defined in the respective lid 120 or fluid collector 110), gravity, and combinations thereof.

The first mating surface 710 is located proximal to the opening plane 220 relative to the second mating surface 720, which allows the first gasket 140 to undergo compression (as shown in FIG. 10A in the Z direction) before the second gasket 150 undergoes compression (as shown in FIG. 10B in the Z direction). Stated differently, the first gasket 140 and first mating surface 710 are configured to engage one another when the lid 120 is affixed to the fluid collector 110, but the second gasket 150 and the second mating surface 720 are configured to not engage one another when the lid 120 is affixed to the fluid collector 110 until later forces are applied to bring the two in contact with one another.

In various embodiments, a stop surface 1030 is included in the fluid collector 110 to help capture the second gasket 150 and to engage with the second mating surface 720 to prevent over-tightening of the lid 120 or over-compression of the gaskets.

Accordingly, an operator may establish a seal for the collection device 100 by compressing the first gasket 140 via a downward force 810 when engaging the lid 120 with the fluid collector 110, but may leave the second gasket 150 uncompressed. Once the seal is established (e.g., via the first gasket 140 as is shown in FIG. 10A), the operator may apply suction to the collection device 100 via the vacuum tubing 190b connected to a pump or vacuum source (not illustrated). This suction applies the additional force to engage the second gasket 150 with the second mating surface 720 once a threshold negative pressure is supplied to the fluid containment region 180, thereby forming a second seal between the lid 120 and the fluid collector 110. In various embodiments, the negative pressure threshold may be selected by the designer for various values for a pressure difference between the external environment and the fluid containment region 180 to be at least 1 PSI less than current atmospheric pressure, between 1 and 5 PSI less than current atmospheric pressure, 5 PSI less than current atmospheric pressure, between 5-10 PSI less than current atmospheric pressure, 10 PSI less than current atmospheric pressure, between 10-15 PSI less than current atmospheric pressure, or 15 PSI less than current atmospheric pressure.

By allowing an operator to establish a first seal without having to compress the second gasket 150, the amount of force required from the operator to establish the first seal is reduced. Additionally, by establishing two seals, the fluid containment region 180 is safeguarded against a loss of seal via one of the gaskets, and the collection device 100 may be subject to greater negative pressures than devices using a single seal.

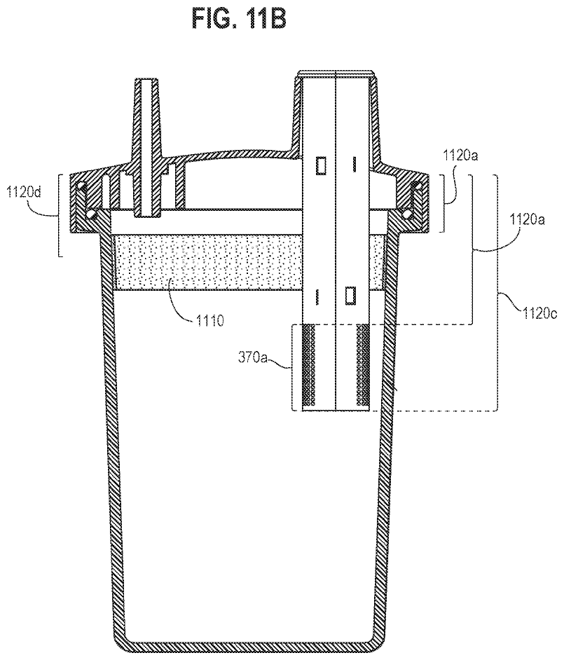

FIGS. 11A and 11B illustrate cutaway views of the collection device 100 that include a pressure film 1110 within the fluid containment region 180, according to embodiments of the present disclosure. In various embodiments, a pressure film 1110 may be secured within the fluid containment region 180 to an inner portion of the wall 112 of the fluid collector 110 by an adhesive tape (double-sided), glue, epoxy, or other adhesive.

In various embodiments, the pressure film 1110 is used to measure the pressure applied to the fluid containment region 180 so that, as pressure is applied, changes in color density are displayed by the pressure film 1110 that correspond to the pressure distribution. The pressure film 1110 may be used to indicate whether the fluid containment region 180 reaches a desired pressure when suction is applied or has maintained a desired pressure for at least a threshold amount of time after suction has been applied (or removed). In various embodiments, the color change in the pressure film 1110 may be reversible (e.g., able to change back to an initial color when the pressure is removed) or irreversible (e.g., permanently showing color associated with the highest/lowest pressure applied to date).

To indicate the pressure within the fluid containment region 180, the pressure film 1110 disposed therein is configured to change color in response to changes in pressure in the fluid containment region 180. As shown in FIG. 11A, the pressure film 1110 exhibits a first color, indicative of a first pressure, and as shown in FIG. 11B, the pressure film 1110 exhibits a second color, different from the first color, indicative of exposure to a given pressure.

In various embodiments, pressure films 1110 may be selected to exhibit various color changes (e.g., from white to black, from red to green) at various pressures, and may change gradually in color as pressure changes, or may change in reaction to experiencing a threshold pressure (e.g., maintaining a steady color until the threshold pressure is reached). In various embodiments, the pressure films 1110 changes color corresponding to the vacuum pressure it is exposed to. When applied to the inside of the collection device 100, the pressure film 1110 gradually changes color as pressure changes, or may change in reaction to experiencing a threshold vacuum pressure (e.g., maintaining a steady color until the threshold vacuum pressure is reached).

In various embodiments, the pressure film 1110 provides the operator with a visual indication of the pressure or pressure changes within the collection device 100, thereby allowing the operator to easily troubleshoot whether any leaks, inconsistent pressures, or unexpected pressures exist within the collection device 100. For example, if the operator believes there is a leak in the collection device 100 but the pressure film 1110 within the collection device 100 shows that a vacuum pressure is being maintained, then the operator need not disassemble the collection device 100 and can troubleshoot other system components, thereby reducing the amount of time spent troubleshooting.

The pressure film 1110 is positioned within the fluid collector 110 to be visible to a viewer without having to remove the lid 120 from the fluid collector 110, and may be visible during operation of the collection device 100. Accordingly, when the lid 120 extends for a first distance 1120a from the first plane 220, the pressure film 1110 is located a second distance 1120b from the first plane 220 to allow for unobstructed viewing. Additionally, when the plurality of fluid outlets 360 in the solids filter 130 are located at least a third distance 1120c from the first plane 220, the second distance 1120b is defined to be less than the third distance 1120c to avoid the fluid 530 contacting the pressure film 1110. Stated differently, the height at which the pressure film 1110 is disposed in the fluid containment region 180 is set to be between the lid 120 and the first portion 370a (or collecting face 350) of the solids filter 130.

Because the solids filter 130 and the return filter 170 extend into the fluids collector 110 through the zone defined by the second distance 1120b, in various embodiments, the pressure film 1110 may extend around the inner circumference of the wall 112 of the fluid collector 110 to ensure visibility. In other embodiments, one or more lengths of the pressure film 1110 may be disposed at different locations around the circumference of the wall 112 within the zone defined by the second distance 1120b to use less of the pressure film 1110, while also ensuring visibility that is not obscured by the solids filter 130 or the return filter 170 (from various angles).

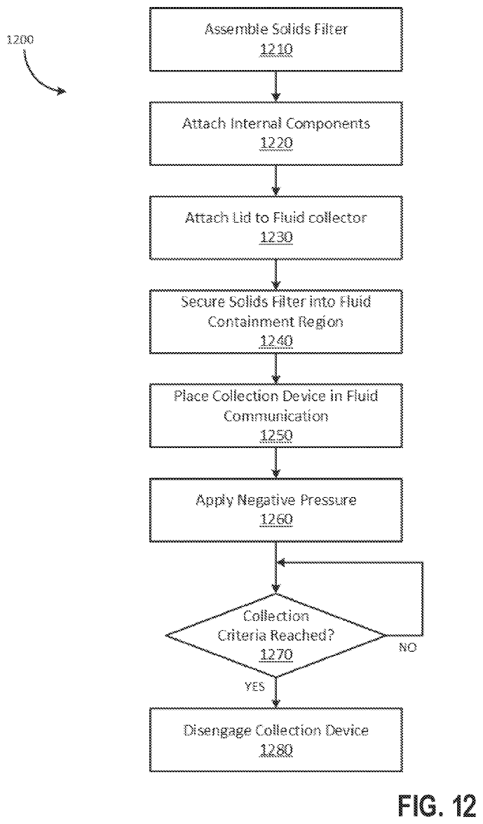

FIG. 12 is a flowchart of a method 1200 for assembling and using a collection device 100, according to embodiments of the present disclosure. Method 1200 begins at block 1210, where a user assembles the solids filter 130. In various embodiments, the user connects two sections 310 together to assembly the solids filter 130, which can include two identical sections 310, or two sections 310 with one or more of different numbers, localizations, orientations, and bore sizes of the fluid outlets 360 included in the respective sections 310. The combined sections 310 thereby provide a solids filtration means for the collection device 100 that a user can customize to provide various flowrates and flow paths for a fluid 530. Additionally, the translucent walls of the sections 310 allows for visual inspection while the solids filtration means is in use to determine whether fluid 530 is flowing through the solids filtration means (and the rate thereof) and whether any solids 540 have been collected by the solids filtration means.

Although presented in FIG. 12 with block 1210 occurring before block 1220, in various embodiments, block 1220 may occur before or substantially simultaneously with block 1210 in method 1200. Similarly, although presented in FIG. 12 with block 1210 occurring before block 1230, block 1230 may occur before or substantially simultaneously with block 1210 in method 1200. Additionally, in some embodiments, block 1210 may be omitted from method 1200 if using a preassembled or single-piece solids filter 130.

At block 1220, the user (optionally) attaches internal components to the pressure containment means before forming an airtight seal. In various embodiments, the internal components includes a pressure indicating means that is affixed to an internal wall of a fluid collector 110 to be used as part of fluid containment region 180. In various embodiments, the pressure indicating means is affixed via an adhesive to the internal wall, but may also be affixed via cutouts, shelves, or pressure clips built into the internal wall.

In various embodiments, the user can select various pressure films 1110 with various sensitives and pressure measurement ranges for use as the pressure indicating means to allow for the user to visually inspect through the translucent wall 112 of the fluid collector 110 whether a desired negative pressure has been reached or maintained during operation of the collection device 100. Additionally, the pressure indicating means may provide the user with an indication for whether the pressure imparted to the fluid containment region 180 has been maintained for at least a predefined amount of time after the fluid collector 110 may be disengaged from a collection target or a negative pressure source to verify that the airtight seals have been maintained.

In various embodiments, during block 1220, the user may additionally or alternatively secure an aerosol filtration means (e.g., a return filter 170) to the lid 120 to reduce intake of any fluid or aerosolized components thereof that collected in the fluid containment region 180 from flowing out of fluid containment region 180 or otherwise being sucked into a negative pressure source while negative pressure is being imparted. In various embodiments, the aerosol filtration means is disposed within the fluid containment region 180 once the lid 120 is selectively engaged to the fluid collector 110.

At block 1230, the user attaches the lid 120 to the fluid collector 110. In various embodiments, the user places the lid 120 in contact with the opening of the fluid collector 110, applies a downward force 810 to seat the lid 120 onto the fluid collector 110, and a rotational force 820 in a first direction to engage a plurality of twist-locks to ensure that an airtight seal is formed and maintained between the lid 120 and the fluid collector 110. In various embodiments, the lid 120 and the fluid collector 110 engage to form a pressure containment means that includes a fluid containment region 180 and at least a portion of the pressure containment means is translucent (e.g., the fluid collector 110) to allow for visual inspection while the pressure containment means is in use.

In various embodiments, the lid 120 and the fluid collector 110 include a first and a second sealing means to form the airtight seal, such as the first gasket 140 and first mating surface 710 and second gasket 150 and second mating surface 720 (described in greater detail in relation to FIGS. 10A and 10B). In various embodiments, the airtight seal is initially formed by the first sealing means (e.g., the first gasket 140 and first mating surface 710), and is later supplemented by the second sealing means when negative pressure draws the lid 120 tighter onto the fluid collector (e.g., during block 1240). Accordingly, a user may applying a downward force 810 and a rotational force 820 to engage a plurality of first engagement means (e.g., acceptors 7410) with a plurality of second engagement means (e.g., tabs 730) to seal the pressure containment means via the first sealing means, but the second sealing means may remain not engaged until a negative pressure is imparted to the pressure containment means via a negative pressure source.

At block 1240, the user secures the solids filter into the fluid containment region 180. In various embodiments, engagement features in the solids filter 130 and the lid 120 position the solids filter 130 at a desired location relative to the walls 112 of the fluid collector 110, which can include an off-center position (e.g., to locate the solids filter 130 closer to one side of the fluid collector 110 for ease of visual inspection). The engagement features can include the relative shapes of the solids filter 130 and corresponding port or through-hole in the lid 120, various filter alignment features 610 or port alignment features 620, such as those discussed in relation to FIGS. 6A and 6B. In various embodiments, the solids filtration means is secured into the fluid containment region via a cap 160 that interfaces with a port included in the lid 120.

At block 1250, the user places the collection device 100 in fluid communication with a negative pressure source and a filtration target. In various embodiments, the fluid communications means include intake tubing 190a and vacuum tubing 190b connected to associated ports or through-holes included in the lid 120 and connected, respectively, to the filtration target (e.g., a blood vessel or port associated with a blood vessel) and the negative pressure source (e.g., a vacuum pump).

At block 1260, the user applies negative pressure, via the fluid communication means, to the filtration target. In various embodiments, the user places a vacuum or other negative pressure source in an operation mode to apply negative pressure or suction to the fluid containment region 180. Because the pressure containment means is disposed and in fluid communication between the negative pressure source and the filtration target, the applied negative pressure causes fluid 530 from the filtration target to be pulled or sucked into the fluid containment region 180. The relative position of the solid filtration means in the flow path of the fluid 530 allows the solid filtration means to capture various solids 540 included in the fluid 530 (e.g., based on the size and location of the fluid outlets 360 relative to the size of the solids 540).

At block 1270, the user confirms whether a collection criteria has been reached. When the collection criteria is reached, method 1200 proceeds to block 1280; otherwise, method 1200 continues until the collection criteria is reached. In various embodiments, the collection criteria include at least one of: a total amount of fluid collected in the fluid containment region 180, whether a solid 540 has been collected in the solids filter 130, an amount of solids 540 collected in the solids filter 130, reaching a desired flowrate of the fluid 530 for a corresponding pressure differential supplied by a negative pressure source, a time limit, or the like.

At block 1280, the user disengages the collection device 100 from the filtration target. In various embodiments, disengaging the collection device 100 includes turning off the negative pressure source and removing the fluid communication means from the pressure containment means. In various embodiments, to maintain the applied negative pressure when the fluid communications means are removed, the user may manually engage one or more seals or valves in the fluid communication path (e.g., at the ports or caps of the fluid collection device 100) before removing the fluid communication means, or the fluid communication path may include various one-way valves or other automated sealants that prevent the flow of air when the fluid communication means are removed without further user intervention.

In various embodiments, after disengaging the collection device 100 from the filtration target, the user may leave the collection device 100 sealed (with the applied negative pressure) for a predefined length of time to observe the collected fluid 530 and any collected solids 540 without exposing the contents to the external environment. Because the walls 112 and solids filter 130 are translucent, the user may periodically observe the state of the collection device 100 (and the contents thereof), which may include a pressure film 1110 to verify the internal pressure of the collection device 100.

In various embodiments, the user may selectively disengage the lid 120 from the fluid collector 110 or selectively disengage the solids filter 130 from the lid 120 (and cap 160) to remove the collected fluid 530 or solids 540 for further examination or use.

Method 1200 may conclude after block 1280 or may restart using some or all of the previously assembled components to continue collecting fluid 530 and/or solids 540 from the filtration target.

The descriptions and illustrations of one or more embodiments provided in this disclosure are intended to provide a thorough and complete disclosure the full scope of the subject matter to those of ordinary skill in the relevant art and are not intended to limit or restrict the scope of the subject matter as claimed in any way. The aspects, examples, and details provided in this disclosure are considered sufficient to convey possession and enable those of ordinary skill in the relevant art to practice the best mode of the claimed subject matter. Descriptions of structures, resources, operations, and acts considered well-known to those of ordinary skill in the relevant art may be brief or omitted to avoid obscuring lesser known or unique aspects of the subject matter of this disclosure. The claimed subject matter should not be construed as being limited to any embodiment, aspect, example, or detail provided in this disclosure unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently.

Having been provided with the description and illustration of the present disclosure, one of ordinary skill in the relevant art may envision variations, modifications, and alternate embodiments falling within the spirit of the broader aspects of the general inventive concept provided in this disclosure that do not depart from the broader scope of the present disclosure.

As used in the present disclosure, a phrase referring to “at least one of” a list of items refers to any set of those items, including sets with a single member, and every potential combination thereof. For example, when referencing “at least one of A, B, or C” or “at least one of A, B, or C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

As used in the present disclosure, the terms “substantially”, “approximately”, “about”, and other relative terms encompass values within +5% of a stated quantity, percentage, or range unless a different approximation is explicitly recited in relation to the state quantity, percentage, or range or if the context of the value indicates that a different approximation would be more appropriate. For example, a value identified as about X % may be understood to include values between 0.95*X % and 1.05*X % or between X−0.05X and X+0.05X percent, but may stop at zero or one hundred percent in various contexts. In another example, a feature described as being substantially parallel or perpendicular to another feature shall be understood to be within ±9 degrees of parallel or perpendicular. Any value stated in relative terms shall be understood to include the stated value and any range or subrange between the indicated or implicit extremes.

As used in the present disclosure, all numbers given in the examples (whether indicated as approximate or otherwise) inherently include values within the range of precision and rounding error for that number. For example, the number 4.5 shall be understood to include values from 4.45 to 4.54, while the number 4.50 shall be understood to include values from 4.495 to 4.504. Additionally, any number or range that explicitly or by context refers to an integer amount (e.g., approximately X users, between about Y and Z states), shall be understood to round downward or upward to the next integer value (e.g., X=1 users, Y−1 and Z+1 states).

The following claims are not intended to be limited to the embodiments shown herein, but are to be accorded the full scope consistent with the language of the claims. Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various aspects described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

1. A collection device, comprising:
a lid including a first gasket and a first mating surface that are oriented concentrically to a central axis; and
a fluid collector including a second gasket and a second mating surface that are oriented concentrically to the central axis, wherein the second gasket is located to engage with the first mating surface and the second mating surface is located to engage with the first gasket when the lid is selectively engaged with the fluid collector.

2. The collection device of claim 1, wherein the first gasket and the first mating surface are configured to engage one another when the lid is affixed to the fluid collector, wherein the second gasket and the second mating surface are configured to not engage one another when the lid is affixed to the fluid collector until a threshold negative pressure is supplied to a fluid containment region defined by the lid and fluid collector.

3. The collection device of claim 1, wherein an outer radius of the second gasket is smaller than an inner radius of the first gasket.

4. The collection device of claim 1, wherein the lid includes an intake port that defines a first through-hole, the collection device further comprising:
a solids filter defining a plurality of fluid outlets and configured to selectively interface with the intake port to dispose the plurality of fluid outlets through the first through-hole and into a fluid containment region defined between the lid and the fluid collector;
intake tubing; and
a cap configured to secure the solids filter in the first through-hole and secure the intake tubing into the solids filter via a one-way valve.

5. The collection device of claim 4, wherein the fluid collector and the solids filter are translucent.

6. A collection device, comprising:
a pressure containment means, having a fluid containment region and wherein at least a portion of the pressure containment means is translucent;
a solids filtration means, disposed within the fluid containment region, wherein the solids filtrations means is translucent;
a first fluid communication means connected to the pressure containment means and separated from the fluid containment region via the solids filtration means; and
a second fluid communication means connected to the pressure containment means and a negative pressure source to impart a negative pressure in the fluid containment region to draw a fluid through the first fluid communication means through the solids filtration means and into the fluid containment region.

7. The collection device of claim 6, further comprising:
an aerosol filtration means, disposed within the fluid containment region and connected to the second fluid communication means to reduce intake of the fluid from the fluid containment region into the second fluid communication means when the negative pressure is being imparted.

8. The collection device of claim 6, wherein the solids filtration means is held in the fluid containment region via engagement features interfacing with a first through-hole in a lid of the pressure containment means, wherein the first through-hole is positioned non-concentrically to a central axis of the pressure containment means to place the solids filtration means off-center relative to the central axis.

9. The collection device of claim 6, wherein the pressure containment means includes a lid and a fluid collector with a first sealing means and a second sealing means.

10. The collection device of claim 9, wherein the lid includes a plurality of first engagement means and the fluid collector includes a plurality of second engagement means that each engage with a corresponding first engagement means of the plurality of first engagement means via a downward force and a rotational force in a first direction applied to the lid when in contact with the fluid collector to seal the pressure containment means via the first sealing means.

11. The collection device of claim 10, wherein the second sealing means is not engaged until the negative pressure imparted to the pressure containment means via the negative pressure source.

12. The collection device of claim 10, wherein each first engagement means of the plurality of first engagement means includes a magnet; and
wherein a catchment included in each second engagement means that captures a corresponding first engagement means when the pressure containment means is sealed includes a ferromagnetic inclusion to magnetically interface with the magnet of the corresponding first engagement means.

13. The collection device of claim 10, wherein each second engagement means of the plurality of second engagement means includes a magnet in a catchment that that captures a corresponding first engagement means when the pressure containment means is sealed; and
wherein each first engagement means of the plurality of first engagement means includes a ferromagnetic inclusion to magnetically interface with the magnet of a corresponding second engagement means when the pressure containment means is sealed.

14. The collection device of claim 10, wherein each first engagement means of the plurality of first engagement means includes a first magnet of a first polarity; and
wherein each second engagement means of the plurality of second engagement means includes a second magnet of a second plurality in a catchment that that captures a corresponding first engagement means when the pressure containment means is sealed to magnetically interface with the first magnet of the corresponding first engagement means.

15. The collection device of claim 6, further comprising:
a pressure indicating means disposed in the fluid containment region and visible from outside of the fluid containment region.
